SEQUENCE LISTING (15 sequences)

SEQ ID NO: 1 - protein, 124 aa, Homo sapiens
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSS

SEQ ID NO: 2 - protein, 108 aa, Homo sapiens
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIK

SEQ ID NO: 3 - protein, 12 aa, Homo sapiens
GDSVSSNSAAWG

SEQ ID NO: 4 - protein, 18 aa, Homo sapiens
RIYYRSKWYNSYAVSVKS

SEQ ID NO: 5 - protein, 12 aa, Homo sapiens
YDWVPKIGVFDS

SEQ ID NO: 6 - protein, 12 aa, Homo sapiens
RASQFISSSYLS

SEQ ID NO: 7 - protein, 7 aa, Homo sapiens
GSSSRAT

SEQ ID NO: 8 - protein, 9 aa, Homo sapiens
QQLYSSPMT

SEQ ID NO: 9 - protein, 454 aa, Homo sapiens
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Homo sapiens
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 372 bp, Homo sapiens
caggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagc

SEQ ID NO: 12 - DNA, 324 bp, Homo sapiens
gatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaag

SEQ ID NO: 13 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 14 - DNA, 1419 bp, Homo sapiens
atggcctgggtgtggaccctgcccttcctgatggccgctgcccagtcagtgcaggcccaggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 15 - DNA, 705 bp, Homo sapiens
atgagcgtgctgacccaggtgctggctctgctgctgctgtggctgaccggcaccagatgcgatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

(19) United States
(12) Patent Application Publication
(10) Pub. No.: US 2022/0356261 A1
(43) Pub. Date: Nov. 10, 2022

(54) TREATMENT FOR SJÖGREN'S SYNDROME

(71) Applicant: NOVARTIS AG, Basel (CH)

(72) Inventors: Irina BALTCHEVA, Oberwil (CH); Wolfgang HUEBER, Basel (CH); Stephen OLIVER, Basel (CH); Olivier PETRICOUL, Horbourg-Wihr (FR)

(21) Appl. No.: 17/773,935

(22) PCT Filed: Oct. 23, 2020
(86) PCT No.: PCT/US2020/057184

§ 371 Date: May 3, 2022

Related U.S. Application Data
(60) Provisional application No. 62/931,292, filed on Nov. 6, 2019.

(51) Int. Cl. C07K 16/28; A61K 39/395; A61K 31/573; A61P 37/06

(52) U.S. Cl. CPC C07K 16/2878; A61K 39/3955; A61K 31/573; A61P 37/06; A61K 39/00

(57) ABSTRACT

The present disclosure relates to methods for treating Sjögren's Syndrome disease using therapeutically effective doses of anti-BAFFR antibody, such as ianalumab.

### TECHNICAL FIELD

The present disclosure relates to methods for treating Sjögren's syndrome (SjS) using an antibody against BAFFR (BAFF receptor), such as ianalumab.

### BACKGROUND OF THE DISCLOSURE

Primary Sjögren's syndrome (pSS) is a common chronic autoimmune disease of unknown etiology. The impact of this disease on quality of life (QoL) measures is substantial and comparative studies indicated that pSS QoL scored quantitatively worse than congestive heart failure or many cancers (Segal et al 2009; Kuenstner et al 2002; Komaroff et al 1996). The mechanism underlying the development of SjS is the destruction of the epithelium of the exocrine glands, as a consequence of autoreactive B cells and T cells (Brito-Zeròn P., et al, (2016) Treating the Underlying Pathophysiology of Primary Sjögren Syndrome: Recent Advances and Future Prospects. Drugs p. 1601-1623). The high prevalence of autoantibodies, especially against Ro/SSA, even at a very early stage suggests that autoreactive B cells participate in the pathomechanism of SjS (Nocturne G., et al, (2018) B cells in the pathogenesis of primary Sjögren syndrome. Nat Rev Rheumatol p. 133-145). Moreover, increased B cell activity in pSS results in an increased risk for malignant transformation with lymphoma development occurring in 5% of pSS patients.

Clinical features of Sjögren's syndrome can be divided into medically evaluable and patient-symptomatic manifestations. At the present time, there is no single assessment tool that can capture disease activity of both these clinical manifestations of SjS. Therefore, the “European League Against Rheumatism (EULAR) Sjögren Syndrome (SS) Patient Reported Index” (ESSPRI) and the EULAR SS Disease Activity Index (ESSDAI) are widely accepted as well as validated, to measure symptomatic and systemic manifestations of SjS (Franceschini F., et al, (2017), BMC Medecine, 15:69).

Treatment for SjS patients is limited to symptomatic care for the mucosal signs and symptoms, and to date no evidence-based, systemic therapy has been available for SjS patients. Glucocorticoids and typical disease-modifying anti-rheumatic drugs (DMARDs) are mostly ineffective, and no pharmacologic intervention is effective against the severe, disabling fatigue. Despite a lack of convincing evidence of efficacy and based on anecdotal evidence as well as experience from similar autoimmune diseases such as systemic lupus erythematosus, antimalarials (Tishler et al 2008), methotrexate (Winzer and Aringer 2010) or azathioprine (Kaufman et al 1999) are sometimes used, in particular for the treatment of extraglandular symptoms such as renal or joint involvement.

Because the pattern of B cell autoreactivity is to some extent similar to systemic lupus and rheumatoid arthritis, recently, B cell depletion therapy using the anti-CD20 monoclonal antibody (mAb) rituximab has been evaluated for both glandular and extra-glandular manifestations of SjS as well as for lymphoma management with varying degree of success. However, this approach is currently not an approved treatment of SjS. The insufficient efficacy of rituximab could be related to incomplete B cell depletion in the affected tissues (Brito-Zeròn P et al (2016) Treating the Underlying Pathophysiology of Primary Sjögren Syndrome: Recent Advances and Future Prospects. Drugs p. 1601-1623).

Despite available treatment for SjS, there remains a high medical need for new treatment options for SjS subjects.

Antibodies against BAFFR are known from e.g. WO 2010/007082 and include antibodies which are characterized by comprising a VH domain with the amino acid sequence of SEQ ID NO: 1 and a VL domain with the amino acid sequence of SEQ ID NO: 2. The antibody MOR6654 is one such antibody (IgG1 kappa). It has the heavy chain amino acid sequence of SEQ ID NO: 9 and the light chain amino acid sequence of SEQ ID NO: 10. This antibody may be expressed from SEQ ID NOs: 14 and 15, preferably in a host cell which lacks fucosyl-transferase, for example in a mammalian cell line with an inactive FUT8(−/−) gene, to provide a functional non-fucosylated anti-BAFFR antibody with enhanced ADCC. This antibody is referred to hereafter as MOR6654B or VAY736, or under its international non-proprietary name ianalumab. Alternative ways to produce non-fucosylated antibodies are known in the art.

### SUMMARY OF THE DISCLOSURE

The aim of the invention is to provide novel method of treating Sjögren's Syndrome disease (also refered to herein as active Sjögren's (syndrome or disease or recommended terminology used by health authorities), or Sjögren's or SjS) in a subject in need of such treatment, comprising administering to said subject, a therapeutically effective amount of an anti-BAFFR antibody, such as ianalumab.

It has been found that human, anti-BAFFR antibody, such as ianalumab are suitable for the treatment of Sjögren's Syndrome disease (SjS). Particularly, the antibody ianalumab has, in clinical study, shown promise of offering a new treatment modality in clinically active SjS. Therefore, disclosed herein are methods of treating SjS, e.g. primary Sjögren's syndrome in a human subject, comprising administering a therapeutically effective dose of anti-BAFFR antibody, such as ianalumab.

### BRIEF DESCRIPTON OF THE FIGURES

FIG. 1 shows, following single dose treatment with ianalumab, trend towards improvement in Parotid/submandibular gland echostructure.

FIG. 2 shows statistically significant dose response relationship for ESSDAI following treatment with ianalumab in patient with primary Sjögren's syndrome.

### DETAILED DESCRIPTION OF THE DISCLOSURE

The BAFFR:BAFF pair is critically involved in the maturation of transitional B-cells, for survival and activation of mature B-cells, and for isotype class switching in response to T cell-dependent antigens. BAFF and its receptor BAFFR (BAFF receptor) are also important for survival and growth of malignant B-cells. Further, BAFFR normally is not expressed on pre-B cells, but was recently shown to be expressed on human ALL (B-lineage acute lymphoblastic leukemia) cells (Parameswaran, 2010, Cancer Res. 70(11) 4346-4356). The removal of autoreactive B cells and the blockade of inappropriate survival/activation mediated by excess BAFF levels in patients. Thus, an anti-BAFFR antibody, in particular an antibody capable of antibody-dependent cell-mediated cytotoxicity (ADCC) and blockade of ligand binding to BAFFR may offer an effective therapeutic agent in Sjögren's syndrome. Both mechanisms are expected to lead to profound B cell depletion in blood and lymphoid organs and tissues or at least to block the BAFF: BAFF-R mediated activation of tissue B cells.

Ianalumab is a human IgG1/K mAb designed to target human BAFF-R and to competitively inhibit binding of BAFF to BAFF-R, thereby blocking BAFF-R-mediated signaling in B cells. In addition, ianalumab was engineered to effectively eliminate B cells from circulation in vivo by ADCC. ADCC activity of ianalumab is greatly enhanced by elimination of fucose residues from the carbohydrate moiety attached to the Fc part of the antibody. Accordingly, ianalumab shows potent ADCC activity in vitro with an EC50 of 2.0 pM. Thus, ianalumab eliminates BAFF-R+mature and immature B cells via dual mechanisms: (1) antibody-dependent cytotoxicity (ADCC) and (2) induction of B cell apoptosis by blocking BAFF:BAFF-R interaction and downstream survival pathway in B cells. BAFF-R expression is limited to immature and mature B cells up to the lymphoblast stage, and thus earlier stage pro-B and pre-B cells are not directly affected by ianalumab.

Accordingly, we have now devised dosing regimens for treating SjS patients with an anti-BAFFR antibody, such as ianalumab.

In one embodiment, an anti-BAFFR antibody is provided, said antibody comprising an immunoglobulin VH domain comprising the amino acid sequence of SEQ ID NO: 9 and an immunoglobulin VL domain comprising the amino acid sequence of SEQ ID NO: 10, and wherein said antibody is to be administered to a subject in need thereof, as a dose of from about 50 mg to about 300 mg.

In a preferred embodiment, an anti-BAFFR designated VAY736 (ianalumab) is provided. Specifically, VAY736 (ianalumab) comprises the heavy chain amino acid sequence of SEQ ID NO: 9 and the light chain amino acid sequence of SEQ ID NO: 10, and wherein said antibody is to be administered to a subject in need thereof, as a dose of from about 50 mg to about 300 mg. In one embodiment, the route of administration is subcutaneous or intravenous of the antibody according to the embodiments herein described, or a combination of subcutaneous or intravenous.

Some patients may benefit from a loading regimen (e.g., weekly for several weeks [e.g., 1 to 5 weeks, e.g., dosing at weeks 0, 1, 2, 3 and/or 4] or biweekly for several weeks (e.g., 2 to 8 weeks, e.g., dosing at weeks 0, 2, 4, and/or 6) followed by maintenance regimen, e.g. a monthly maintenance regimen. For example, an appropriate regimen for anti-BAFFR antibody can be weekly or bi-weekly for several weeks [e.g., 1 to 5 weeks, e.g., dosing at weeks 0, 1, 2, 3 and/or 4] followed by a monthly maintenance regimen.

In another example, an appropriate regimen for ianalumab is biweekly for several weeks (e.g., 2 to 8 weeks, e.g., dosing at weeks 0, 2, 4, and/or 6) followed by a monthly maintenance regimen.

In some embodiments, the anti-BAFFR antibody, such as ianalumab, may be administered to the patient at an initial dose of 300 mg delivered s.c., and the dose may be then adjusted if needed, as determined by a physician.

In yet another specific embodiment, a dose which comprises two unit doses of 150 mg ianalumab is administered s.c. every four (4) weeks (q4w).

Ianalumab may be administered quarterly, monthly, weekly or biweekly e.g. subcutaneously at a dosing of about 50 mg to 500 mg, e.g. about 150mg to about 400mg, e.g. about 150 mg to about 300 mg, or a e.g. about 200 mg to about 300 mg being administered, by subcutaneous injection, at an unit dose of about 50 mg, about 100 mg, about 150 mg, about 200 mg or about 300 mg.

Ianalumab may be administered by subcutaneous injection, bi-weekly, or monthly at a dose of about 50 mg to about 300 mg, preferably about 300 mg.

As herein defined, “unit dose” refers to a s.c. dose that can be comprised between about 50 mg to 500 mg, e.g. about 150 mg to about 400 mg, e.g. about 150 mg to about 300 mg, or a e.g. about 200 mg to about 300 mg. For example an unit S.C. dose is about 50 mg, about 150 mg, about 150 mg, about 200 mg, about 250 mg, about 300 mg.

In one embodiment, the present invention comprises administering ianalumab to a patient with SjS, in the range of about 20 mg to about 500 mg per treatment, preferably in the range of 30 mg to 300 mg, preferably in the range of 100mg to 300mg, perferably 150 mg to 300 mg per treatment. In one embodiment a patient recieves 20 mg to 300 mg per treatment. In one embodiment patient recieves 150 mg to 300 mg per treatment. In one embodiment patient recieves 20 mg, 30 mg, 60 mg, 90 mg, 120 mg, 150 mg, 180 mg, 200 mg, 210 mg, 250 mg, 275 mg, or 300 mg per treatment. In one embodiment the patient with SjS, receives each treatment every 2 weeks, every 3 weeks, monthly (every 4 weeks), every 6 weeks, bimonthly (every 2 months), every 9 weeks or quarterly (every 3 months). In one embodiment the patient receives each treatment every 3 weeks. In one embodiment the patient receives each treatment every 4 weeks.

When safety concern raises, the dose can be down-titrated, preferably by increasing the dosing interval, preferably by doubling or tripling the dosing interval. For example 300mg monthly or every 3 weeks regimen can be doubled to every 2 month or every 6 weeks respectively or tripled to every 3 month or every 9 weeks respectively.

In some embodiments, the anti-BAFFR antibody, such as ianalumab, may refer to antibodies which have demonstrated to be biosimilar to or interchangeable to ianalumab. Those antibodies may be administered according the embodiments which refer to ianalumab administration, as herein disclosed.

Definitions:

For purposes of interpreting this specification, the following definitions will apply and whenever appropriate, terms used in the singular will also include the plural and vice versa.

The term “antibody” as referred to herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three or four domains, depending on the isotype, C_{H}1, C_{H}2, C_{H}3 and C_{H}4. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., a portion of BAFFR). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., 1989 Nature 341:544-546), which consists of a VH domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities, e.g., an isolated antibody that specifically binds human BAFFR is substantially free of antibodies that specifically bind antigens other than BAFFR. An isolated antibody that specifically binds BAFFR may, however, have cross-reactivity to other antigens, such as BAFFR molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align. Throughout this specification, the complementarity determining region (“CDR”) is defined according to the any of the above mentioned schemes.

The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM, IgA, IgD, IgE and IgG such as IgG1, IgG2, IgG3 or IgG4) that is provided by the heavy chain constant region genes.

The phrases “an antibody recognizing an antigen” and “an antibody specific for an antigen” are used interchangeably herein with the term “an antibody which binds specifically to an antigen”. As used herein, an antibody that “specifically binds to BAFFR polypeptide” or an “anti-BAFFR antibody” refers to an antibody that binds to human BAFFR polypeptide of SEQ ID NO: 13 with a K_{D} of 100 nM or less, 10 nM or less, 1 nM or less. An antibody that “cross-reacts with an antigen other than BAFFR” refers to an antibody that binds that antigen with a K_{D} of 0.5×10⁻⁸ M or less, 5×10⁻⁹ M or less, or 2×10⁻⁹ M or less. An antibody that “does not cross-react with a particular antigen” is intended to refer to an antibody that binds to that antigen, with a K_{D} of 1.5×10⁻⁸ M or greater, or a K_{D} of 5-10×10⁻⁸ M or 1×10⁻⁷ M or greater. In certain embodiments, such antibodies that do not cross-react with the antigen exhibit essentially undetectable binding against these proteins in standard binding assays.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutical combination” as used herein means a product that results from the use or mixing or combining of more than one active ingredient. It should be understood that pharmaceutical combination as used herein includes both fixed and non-fixed combinations of the active ingredients.

The terms “co-administration” or “combined administration” or the like as utilized herein are meant to encompass the administration of one or more compounds described herein together with a selected combination partner to a single subject in need thereof (e.g., a patient or subject), and are intended to include treatment regimens in which the compounds are not necessarily administered by the same route of administration and/or at the same time.

The term “pharmaceutical composition” is defined herein to refer to a mixture (e.g., a solution or an emulsion) containing at least one active ingredient or therapeutic agent to be administered to a warm-blooded animal, e.g., a mammal or human, in order to prevent or treat a particular disease or condition affecting the warm-blooded animal.

The term “a therapeutically effective amount” of a compound of the present disclosure refers to an amount of the compound of the present disclosure that will elicit the biological or medical response of a subject (patient of subject), for example, reduction or inhibition of an enzyme or a protein activity, or ameliorate symptoms, alleviate conditions, slow or delay disease progression, or prevent a disease, etc. The therapeutically effective dosage of a compound, the pharmaceutical composition, or the combinations thereof, is dependent on the species of the patient, the body weight, age, sex, and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The phrase “therapeutic regimen” means the regimen used to treat an illness, e.g., the dosing protocol used during the treatment of pSS. A therapeutic regimen may include an induction regimen and a maintenance regimen.

The term “dosing”, as used herein, refers to the administration of a substance (e.g., an anti-BAFFR antibody) to achieve a therapeutic objective (e.g., the treatment of a SjS).

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In a preferred embodiment, the subject is a human. The term “subject” is used interchangeably with “patient” when it refers to human.

As used herein, a subject is “in need of” a treatment if such subject would benefit biologically, medically or in quality of life from such treatment.

As used herein, the phrase “population of patients” is used to mean a group of patients.

The term “comprising” encompasses “including” as well as “consisting,” e.g., a composition “comprising” X may consist exclusively of X or may include something additional, e.g., X+Y. AUCO-t designates the area under the plasma concentration-time curve from time zero to time T where t is a defined time point after administration [mass×time/volume].

AUCtx-ty represents the area under the plasma concentration-time curve from time ‘x’ to time ‘y’ where ‘time x’ and ‘time y’ are defined time points after administration. Cmax is the observed maximum plasma concentration following drug administration [mass/volume]. Cmin is the observed minimum plasma concentration following drug administration Ctrough is the observed plasma concentration that is just prior to the beginning of, or at the end of a dosing interval.

Tmax is the time to reach the maximum concentration after drug administration [time]. ss (subscript) indicate that the parameter is defined at steady state.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “treatment” or “treat” is herein defined as the application or administration of a compound according to the disclosure, (compound of Formula (I), or a pharmaceutically acceptable salt thereof, or a pharmaceutical composition comprising said compound, to a subject or to an isolated tissue or cell line from a subject, where the subject has a particular disease (e.g., SjS), a symptom associated with the disease (e.g., SjS), or a predisposition towards development of the disease (e.g., SjS) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By “selecting”, “selectively treating” and “selectively administering”, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having SjS.

Sjorgren Syndrome and Effectiveness of Treatment According to the Invention

The disclosed anti-BAFF antibody, i.e., ianalumab, may be used in vitro, ex vivo, or incorporated into pharmaceutical compositions and administered in vivo to treat SjS patients (e.g., human patients).

The effectiveness of a Sjögren's treatment may be assessed using various known methods and tools that measure Sjögren's Syndrome state and/or Sjögren's clinical response. Some examples include, e.g., EULAR Sjögren's Syndrome Disease Activity Index (ESSDAI), Physician Global Assessment Scale (PhGA), EULAR Sjögren's Syndrome Patient Reported Index (ESSPRI), The Functional Assessment of Chronic Illness Therapy-Fatigue Scale (FACIT-Fatigue) and EQ5D.

Efficacy

Clinical efficacy measurements related to primary and secondary objectives are outlined below.

EULAR Sjögren's Syndrome Disease Activity Index (ESSDAI)

ESSDAI is a validated disease outcome measure for Sjögren's Syndrome and is applied to the study subjects (Seror R, et al (2015) Validation of EULAR primary Sjögren's syndrome disease activity (ESSDAI) and patient indexes (ESSPRI). Ann. Rheum. Dis. p. 859-66). The instrument contains 12 organ-specific domains contributing to disease activity. For each domain, features of disease activity are scored in 3 or 4 levels according to their severity. These scores are then summed across the 12 domains in a weighted manner to provide the total score. The domains (weights) are as follows: constitutional (3), lymphadenopathy (4), glandular (2), articular (2), cutaneous (3), pulmonary (5), renal (5), muscular (6), PNS (5), CNS (5), hematological (2), and biological (1). The maximum possible score is 123.

To calculate ESSDAI, all 12 organ domains must be individually assessed at every scheduled timepoint (from screening visit till end of study). Domain assessments are entered into a tablet (provided by a central vendor) and ESSDAI score is calculated by the software. For assessments not listed in the protocol as mandatory tests but which may be needed to estimate ESSDAI, including radiography, high resolution computer tomography (HRCT), lung function test (DLCO, FVC), estimated glomerular filtration rate (eGFR), electromyography (EMG), muscle (or any other) biopsy, it is at the investigator's discretion to have these assessed based on the signs and symptoms of the patient so to provide correct ESSDAI readout. The EULAR Sjorgen syndrome disease index (ESSDAI), domain and item definitions and weights are summarized in the table below:

| | Activity | |
|---|---|---|
| Domain [weight] | level | Description |
| Constitutional [3] | No = 0 | Absence of the following |
| Exclusion of fever of | Low = 1 | symptoms |
| infectious origin and | Moderate = 2 | Mild or intermittent fever |
| voluntary weight loss | | (37.5-38.5° C.)/night sweats |
| | | and/or involuntary weight loss |
| | | of 5-10% of body weight |
| | | Severe fever (>38.5° C.)/night |
| | | sweats and/or involuntary |
| | | weight loss of >10% of body |
| | | weight |
| Lymphadenopathy [4] | No = 0 | Absence of the following |
| Exclusion of infection | Low = 1 | features |
| | Moderate = 2 | Lymphadenopathy ≥1 cm in |
| | High = 3 | any nodal region or ≥2 cm in |
| | | inguinal region |
| | | Lymphadenopathy ≥2 cm in |
| | | any nodal region or ≥3 cm in |
| | | inguinal region, and/or |
| | | splenomegaly (clinically |
| | | palpable or assessed by |
| | | imaging) |
| | | Current malignant B-cell |
| | | proliferative disorder |
| Glandular [2] | No = 0 | Absence of glandular |
| Exclusion of stone or | Low = 1 | swelling |
| infection | Moderate = 2 | Small glandular swelling with |
| | | enlarged parotid (≤3 cm), or |
| | | limited submandibular or |
| | | lachrymal swelling |
| | | Major glandular swelling with |
| | | enlarged parotid (>3 cm), or |
| | | important submandibular or |
| | | lachrymal swelling |
| Articular [2] | No = 0 | Absence of currently active |
| Exclusion of | Low = 1 | articular involvement |
| osteoarthritis | Moderate = 2 | Arthralgias in hands, wrists, |
| | High = 3 | ankles and feet accompanied |
| | | by morning stiffness |
| | | (>30 min) |
| | | 1-5 (of 28 total count) |
| | | synovitis |
| | | ≥6 (of 28 total count) |
| | | synovitis |
| Cutaneous [3] | No = 0 | Absence of currently active |
| Rate as ‘no activity’ | Low = 1 | cutaneous involvement |
| stable long-lasting | Moderate = 2 | Erythema multiforma |
| features related | High = 3 | Limited cutaneous vasculitis, |
| to damage | | including urticarial vasculitis, |
| | | or purpura limited to feet and |
| | | ankle, or subacute lutaneous |
| | | lupus |
| | | Diffuse cutaneous vasculitis, |
| | | including urticarial vasculitis, |
| | | or diffuse purpura, or ulcers |
| | | related to vasculitis |
| Pulmonary* [5] | No = 0 | Absence of currently active |
| Rate as ‘no activity’ | Low = 1 | pulmonary involvement |
| stable long-lasting | Moderate = 2 | Persistent cough or bronchial |
| features related to | High = 3 | involvement with no |
| damage, or respiratory | | radiographic abnormalities on |
| involvement not related | | radiography or radiological or |
| to the disease | | HRCT evidence of interstitial |
| (tobacco use, etc) | | lung disease with no |
| | | breathlessness and normal |
| | | lung function test |
| | | Moderately active pulmonary |
| | | involvement, such as |
| | | interstitial lung disease |
| | | shown by HRCT with |
| | | shortness of breath on |
| | | exercise (NYHA II) or |
| | | abnormal lung function tests |
| | | restricted to 70% > DL_{co} ≥ 40% |
| | | or 80% > FVC ≥ 60% |
| | | Highly active pulmonary |
| | | involvement, such as |
| | | interstitial lung disease |
| | | shown by HRCT with |
| | | shortness of breath at rest |
| | | (NHYA III, IV) or with |
| | | abnormal lung function tests |
| | | DL_{co} < 40% or FVC < 60% |
| Renal [5] | No = 0 | Absence of currently active |
| Rate as ‘no activity’ | Low = 1 | renal involvement with |
| stable long-lasting | Moderate = 2 | proteinuria <0.5 g/day, no |
| features related to | High = 3 | haematuria, no leucocyturia, |
| damage and renal | | no acidosis, or long-lasting |
| involvement not related | | stable proteinuria due to |
| to the disease. | | damage |
| If biopsy has been | | Evidence of mild active renal |
| performed, please rate | | involvement, limited to |
| activity based on | | tubular acidosis without renal |
| histological features | | failure or glomerular |
| first | | involvement with proteinuria |
| | | (between 0.5 and 1 g/day) |
| | | and without haematuria or |
| | | renal failure (GFR ≥60 |
| | | ml/min) |
| | | Moderately active renal |
| | | involvement, such as tubular |
| | | acidosis with renal failure |
| | | (GFR <60 ml/min) or |
| | | glomerular involvement with |
| | | proteinuria between 1 and |
| | | 1.5 g/day and without |
| | | haematuria or renal failure |
| | | (GFR ≥60 ml/min) or |
| | | histological evidence of extra- |
| | | membranous |
| | | glomerulonephritis or |
| | | important interstitial lymphoid |
| | | infiltrate |
| | | Highly active renal |
| | | involvement, such as |
| | | glomerular involvement with |
| | | proteinuria >1.5 g/day or |
| | | haematuria or renal failure |
| | | (GFR <60 ml/min), or |
| | | histological evidence of |
| | | proliferative |
| | | glomerulonephritis or |
| | | cryoglobulinaemia-related |
| | | renal involvement |
| Muscular* [6] | No = 0 | Absence of currently active |
| Exclusion of weakness | Low = 1 | muscular involvement |
| due to corticosteroids | Moderate = 2 | Mild active myositis shown by |
| | High = 3 | abnormal EMG or biopsy with |
| | | no weakness and creatine |
| | | kinase (N < CK ≤ 2N) |
| | | Moderately active myositis |
| | | confirmed by abnormal EMG |
| | | or biopsy with weakness |
| | | (maximal deficit of 4/5), or |
| | | elevated creatine kinase |
| | | (2N < CK ≤ 4N) |
| | | Highly active myositis shown |
| | | by abnormal EMG or biopsy |
| | | with weakness (deficit ≤3/5) |
| | | or elevated creatine kinase |
| | | (>4N) |
| PNS* [5] | No = 0 | Absence of currently active |
| Rate as ‘no activity’ | Low = 1 | PNS involvement |
| stable long-lasting | Moderate = 2 | Mild active peripheral |
| features related to | High = 3 | nervous system involvement, |
| damage or PNS | | such as pure sensory axonal |
| involvement not | | polyneuropathy shown by |
| related to the disease | | NCS or trigeminal (V) |
| | | neuralgia |
| | | Moderately active peripheral |
| | | nervous system involvement |
| | | shown by NCS, such as |
| | | axonal sensorimotor |
| | | neuropathy with maximal |
| | | motor deficit of 4/5, pure |
| | | sensory neuropathy with |
| | | presence of cryoglobulinamic |
| | | vasculitis, ganglionopathy |
| | | with symptoms restricted to |
| | | mild/moderate ataxia, |
| | | inflammatory demyelinating |
| | | polyneuropathy (CIDP) with |
| | | mild functional impairment |
| | | (maximal motor deficit of 4/5 |
| | | or mild ataxia) Or cranial |
| | | nerve involvement of |
| | | peripheral origin (except |
| | | trigeminal (V) neuralgia) |
| | | Highly active PNS |
| | | involvement shown by NCS, |
| | | such as axonal sensorimotor |
| | | neuropathy with motor deficit ≤3/5, |
| | | peripheral nerve |
| | | involvement due to vasculitis |
| | | (mononeuritis multiplex, etc), |
| | | severe ataxia due to |
| | | ganglionopathy, inflammatory |
| | | demyelinating |
| | | polyneuropathy (CIDP) with |
| | | severe functional impairment: |
| | | motor deficit ≤3/5 or severe |
| | | ataxia |
| CNS* [5] | No = 0 | Absence of currently active |
| Rate as ‘no activity’ | High = 3 | CNS involvement |
| stable long-lasting | | Highly active CNS features, |
| features related to | | such as cerebral vasculitis |
| damage or CNS | | with cerebrovascular accident |
| involvement not | | or transient ischaemic attack, |
| related to the disease | | seizures, transverse myelitis, |
| | | lymphocytic meningitis, |
| | | multiple sclerosis-like |
| | | syndrome with motor deficit |
| Haematological [2] | No = 0 | Absence of auto-immune |
| For anaemia, neutropenia, | Low = 1 | cytopenia |
| and thrombopenia, only | Moderate = 2 | Cytopenia of auto-immune |
| autoimmune cytopenia | High = 3 | origin with neutropenia |
| must be considered | | (1000 < neutrophils < 1500/mm |
| Exclusion of vitamin or | | 3), and/or anaemia |
| iron deficiency, drug- | | (10 < haemoglobin < 12 g/dl), |
| induced cytopenia | | and/or thrombocytopenia |
| | | (100000 < platelets < 150000/m |
| | | m3) Or lymphopenia |
| | | (500 < lymphocytes < 1000/mm |
| | | 3) |
| | | Cytopenia of auto-immune |
| | | origin with neutropenia |
| | | (500 ≤ neutrophils ≤ 1000/mm3), |
| | | and/or anaemia |
| | | (8 ≤ haemoglobin ≤ 10 g/dl), |
| | | and/or thrombocytopenia |
| | | (50000 ≤ platelets ≤ 100000/m |
| | | m3) Or lymphopenia |
| | | (≤500/mm3) |
| | | Cytopenia of auto-immune |
| | | origin with neutropenia |
| | | (neutrophils <500/mm3), |
| | | and/or or anaemia |
| | | (haemoglobin <8 g/dl) and/or |
| | | thrombocytopenia (platelets |
| | | <50000/mm3) |
| Biological [1] | No = 0 | Absence of any of the |
| | Low = 1 | following biological features |
| | Moderate = 2 | Clonal component and/or |
| | | hypocomplementaemia (low |
| | | C4 or C3 or CH50) and/or |
| | | hypergammaglobulinaemia or |
| | | high IgG level between 16 |
| | | and 20 g/l |
| | | Presence of |
| | | cryoglobulinaemia and/or |
| | | hypergammaglobulinaemia or |
| | | high IgG level >20 g/l, and/or |
| | | recent onset |
| | | hypogammaglobulinaemia or |
| | | recent decrease of IgG level |
| | | (<5 g/l) |

Physician Global Assessment Scale (PhGA)

The physician's global assessment scale is used by the Investigator to rate the disease activity of their patient using 100 mm VAS ranging from “no disease activity” (0) to “maximal disease activity” (100).

To enhance objectivity, the physician must not be aware of the specific patient's reported outcome assessments, when performing his own assessment on that patient. Therefore this assessment must be done prior to viewing the patient's global assessment of overall disease activity score.

EULAR Sjögren's Syndrome Patient Reported Index (ESSPRI)

ESSPRI is an established disease outcome measure for Sjögren's Syndrome (Seror R, et al (2011) EULAR Sjögren's Syndrome Patient Reported Index (ESSPRI): development of a consensus patient index for primary Sjögren's syndrome. Ann. Rheum. Dis. p. 968-72). It consists of three of domains of dryness, pain and fatigue. The subject can assess severity of symptoms they experience on a single 0-10 numerical scale for each of the three domains. The ESSPRI score is defined as mean of scores from the three scales: (dryness+pain+fatigue)/3.

FACIT-Fatigue

The Functional Assessment of Chronic Illness Therapy-Fatigue Scale (FACIT-F v4) is a short, 13-item, easy-to-administer tool that measures an individual's level of fatigue during their usual daily activities over the past week. The level of fatigue is measured on a 5-point Likert scale (0=not at all, 1=a little bit, 2=somewhat, 3=quite a bit, 4=very much) (Webster K, et al. (2003) The Functional Assessment of Chronic Illness Therapy (FACIT) Measurement System: properties, applications, and interpretation. Health Qual Life Outcomes p. 79).

EQ5D

EQ-5D is a standardized instrument which measures the health-related quality of life.

The EQ-5D consists of a descriptive system and the EQ VAS scale.

The descriptive system comprises five dimensions: mobility, self-care, usual activities, pain/discomfort and anxiety/depression. This can be used as a quantitative measure of health outcome that reflects the patient's own judgement. The scores on these five dimensions can be presented as a health profile or can be converted to a single summary index number (utility) reflecting preferability compared to other health profiles.

The EQ VAS records the patient's self-rated health on a vertical visual analogue scale with 0 representing ‘Worst imaginable Health State’ and 100 ‘Best imaginable Health State’.

Appropriateness of Efficacy Assessments

Efficacy measures in this study are primarily based on ESSDAI (EULAR SS Disease Activity Index) measuring organ-specific disease criteria, and on ESSPRI (European League Against Rheumatism [EULAR] Sjögren Syndrome [SS] Patient Reported Index) measuring the patient's subjective disease impact. Both instruments are widely accepted and validated, gold-standard measures of systemic and symptomatic manifestations of SjS, respectively.

ESSDAI is a systemic disease activity index that classifies disease activity in 3-4 levels, over each of 12 differentially weighted domains (biologic, hematologic, articular, glandular, cutaneous, constitutional, lymphadenopathy, renal, pulmonary, PNS, CNS and muscular).

A composite weighted score provides an accurate assessment of disease activity, with a good sensitivity to change, as validated in multiple cohort studies (Seror R et al (2015) Validation of EULAR primary Sjögren's syndrome disease activity (ESSDAI) and patient indexes (ESSPRI). Ann. Rheum. Dis. p. 859-66). The ESSPRI tool, on the other hand, is a patient reported composite score of symptoms of dryness, limb pain and fatigue evaluated on 0-10 visual analog scale, during the preceding 2 weeks (Seror R et al (2011) EULAR Sjögren's Syndrome Patient Reported Index (ESSPRI): development of a consensus patient index for primary Sjögren's syndrome. Ann. Rheum. Dis. p. 968-72). Patient reported scores have poor sensitivity to change in disease activity, but among available tools, ESSPRI has been reported to have significantly better sensitivity. A recent prospective study reported poor correlation between systemic and patient scores, suggesting that the two indices evaluate complementary components of disease activity, therefore underscoring the importance of evaluation of both parameters to arrive at an accurate assessment of disease activity and change thereof (Seror R et al (2015) Validation of EULAR primary Sjögren's syndrome disease activity (ESSDAI) and patient indexes (ESSPRI). Ann. Rheum. Dis. p. 859-66).

Pharmaceutical Composition

Pharmaceutical compositions for use in the disclosed methods may be manufactured in conventional manner. Exemplary pharmaceutical composition comprising the anti-BAFFR antibody, such as ianalumab are disclosed in WO 2012/076670 and WO 2013/186700, incorporated herein by reference. In one embodiment, the pharmaceutical composition is provided for administration typically by infusion or via a delivery device (e.g. a syringe) including a pharmaceutical composition of the invention (e.g., pre-filled syringe).

Combinations:

While it is understood that the disclosed methods provide for the treatment of Sjögren's patients, the therapy is not necessarily a monotherapy. Indeed, if a patient is selected for the treatment with an anti-BAFFR antibody, such as ianalumab, then the anti-BAFFR antibody, such as ianalumab, may be administered in accordance with the methods of the disclosure either alone or in combination with other agents and therapies for treating Sjögren's patients, e.g., in combination with at least one additional Sjögren's agent.

Various therapies may be beneficially combined with the disclosed anti-BAFFR antibody, such as ianalumab, during treatment of SjS. Such therapies include steroids (corticosteroid such as prednisone or equivalent); DMARDSs such as for example hydroxychloroquine (Plaquenil), methotrexate (Trexall), sulfasalazine (Azulfidine), minocycline (Minocin) or leflunomide (Arava)); or B-cell depleting drug such as Rituximab.

A skilled artisan will be able to discern the appropriate dosages of the above SjS agents for co-delivery with the disclosed anti-BAFFR antibody, such as ianalumab.

### Embodiments

Methods of Treatment

A1. A method of treating or preventing Sjögren's syndrome, e.g. primary Sjögren's syndrome, in a subject in need thereof, comprising administering to said subject a therapeutically effective amount of an anti-BAFFR antibody or a functional fragment thereof.

A2. A method of treating or preventing Sjögren's syndrome, e.g. primary Sjögren's syndrome, in a subject in need thereof, comprising administering to said subject a therapeutically effective amount of an anti-BAFFR antibody or a functional fragment thereof, wherein the anti-BAFFR antibody or functional fragment thereof includes heavy chain CDR1, CDR2 and CDR3 of SEQ ID NOs 3, 4 and 5 respectively, and light chain CDR1, CDR2 and CDR3 of SEQ ID NOs: 6, 7 and 8.

A3. The method according to Embodiment Al or A2, wherein the anti-BAFFR antibody or functional fragment thereof includes a heavy chain sequence of SEQ ID NO: 9 and a light chain sequence of SEQ ID NO: 10.

A4. The method according to any one of the preceding embodiments, wherein the anti-BAFFR antibody or functional fragment thereof is ianalumab.

A5. The method according to any one of the preceding embodiments, wherein the anti-BAFFR antibody or functional fragment thereof is to be administered as a dose of from about 50 mg to about 300 mg.

A6. The method according to any one of the preceding embodiments, wherein the anti-BAFFR antibody or functional fragment thereof is administered as a dose of about 150 mg to about 300 mg.

A7. The method according to embodiment A6, wherein the anti-BAFFR antibody or functional fragment thereof is administered at about 300 mg.

A8. The method according to embodiment A6, wherein the anti-BAFFR antibody or functional fragment thereof is administered at about 200 mg.

A9. The method according to embodiment A6, wherein the anti-BAFFR antibody or functional fragment thereof is administered at about 150 mg.

A10. The method according to any one of the preceding embodiments, wherein the anti-BAFFR antibody or functional fragment thereof is to be administered subcutaneously.

A11. The method according to any one of the preceding embodiments, wherein the anti-BAFFR antibody or functional fragment thereof is to be administered on a monthly dosing regimen.

Al2. The method according to any one of the preceding embodiments, wherein the anti-BAFFR antibody or functional fragment thereof is to be administered at a dose which comprises two unit doses of 150 mg ianalumab, and which is administered s.c. every four (4) weeks (q4w).

A13. The method according to any one of the preceding embodiments, wherein the anti-BAFFR antibody or functional fragment thereof is to be administered in combination with an additional therapeutic agent.

A14. The method according to any one of the preceding embodiments, wherein the additional therapeutic agent is a steroid, e.g. a corticosteroid.

A15. The method according to embodiment A14, wherein the additional therapeutic agent is prednisone.

A16. The method according to any one of the preceding embodiments, wherein the anti-BAFFR antibody or functional fragment thereof is to be administered in combination with 50 mg prednisone, e.g. wherein prednisone is to be administered orally.

A17. The method according to any one of embodiments All to A16, wherein said subject achieves a sustained response as measured by ESSPRI or ESSDAI after the treatment with the anti-BAFFR antibody or functional fragment thereof.

A18. The method according to any one of preceding embodiments, wherein said antibody is an antibody which has demonstrated to be biosimilar to, or interchangeable to ianalumab.

Isolated Human Anti-BAFFR Antibody

B1: An isolated human anti-BAFFR antibody for use in treating or preventing Sjögren's syndrome (SjS) in a subject in need thereof, wherein the anti-BAFFR antibody is to be administered to said subject from about 50 mg to about 300 mg.

B2. An isolated human anti-BAFFR antibody for use in treating or preventing Sjögren's syndrome (SjS) in a subject in need thereof and wherein said antibody is to be administered subcutaneously to said subject, as a dose of from about 50 mg to about 300 mg.

B4. An isolated human anti-BAFFR antibody for use in treating or preventing Sjögren's syndrome (SjS) in a subject in need thereof, wherein the anti-BAFFR antibody wherein the antibody is to be administered subcutaneously to said subject on a monthly dosing regimen of about 50 mg to about 300 mg, every four weeks, wherein the antibody includes heavy chain CDR1, CDR2 and CDR3 of SEQ ID Nos: 3, 4 and 5 respectively, and light chain CDR1, CDR2 and CDR3 of SEQ ID NOs: 6, 7 and 8.

B5. An isolated human anti-BAFFR antibody, for use in treating or preventing Sjögren's syndrome (SjS) in a subject in need thereof, wherein the antibody is to be administered subcutaneously to the subject on a monthly dosing regimen of 150 mg every 4 weeks, and wherein the antibody includes heavy chain CDR1, CDR2 and CDR3 of SEQ ID Nos: 3, 4 and 5 respectively, and light chain CDR1, CDR2 and CDR3 of SEQ ID NOs: 6, 7 and 8.

B6. An isolated human the anti-BAFFR antibody, for use in treating or preventing Sjögren's syndrome (SjS) in a subject in need thereof, wherein the antibody is to be administered subcutaneously to the subject on a monthly dosing regimen of 300 mg every 4 weeks, wherein the antibody includes heavy chain CDR1, CDR2 and CDR3 of SEQ ID Nos: 3, 4 and 5 respectively, and light chain CDR1, CDR2 and CDR3 of SEQ ID NOs: 6, 7 and 8, and wherein the antibody is to be administered in combination with steroids, e.g. corticosteroids.

B7. The isolated antibody according to embodiment B1 or B2, wherein the antibody the includes a heavy chain sequence of SEQ ID NO: 9 and a light chain sequence of SEQ ID NO: 10.

B8. The isolated antibody according to any one of the preceding embodiments B1 to B7, wherein the antibody is ianalumab, or is an antibody which has demonstrated to be biosimilar to, or interchangeable to ianalumab.

B9. The isolated anti-BAFFR according to embodiment B8, wherein the antibody is to be administered in combination with 50 mg prednisone, optionally wherein prednisone is to be administered orally.

Antibody for Use

C1. An anti-BAFFR antibody or a functional fragment thereof for use in the treatment or prevention of Sjögren's syndrome (SjS) in a subject in need thereof, wherein the anti-BAFFR antibody is to be administered to said subject at a therapeutically effective amount.

C2. An anti-BAFFR antibody or a functional fragment thereof for use in the treatment or prevention of Sjögren's syndrome (SjS) in a subject in need thereof, wherein the anti-BAFFR antibody is ianalumab and wherein ianalumab is to be administered to said subject at a therapeutically effective amount.

C3. An anti-BAFFR antibody or a functional fragment thereof for use in the treatment or prevention of Sjögren's syndrome (SjS) in a subject in need thereof, wherein the anti-BAFFR antibody or a functional fragment thereof is ianalumab and wherein ianalumab is to be administered to said subject at a dose of from about 50 mg to about 300 mg.

C4. An anti-BAFFR antibody or a functional fragment thereof for use in the treatment of Sjögren's syndrome (SjS) in a subject in need of such treatment, wherein the anti-BAFFR antibody is ianalumab and wherein the therapeutic effective amount of ianalumab is about 300 mg.

C5. The anti-BAFFR antibody or functional fragment thereof for use according to any one of embodiments Cl to C3, wherein the dose of said antibody is of about 150 mg.

C6. The anti-BAFFR antibody or functional fragment thereof for use according to any one of embodiments Cl to C3, wherein the dose of said antibody is of about 200 mg.

C7. The anti-BAFFR antibody or functional fragment thereof for use according to any one of embodiments C1 to C3, wherein the dose of said antibody is of about 300 mg.

C8. The anti-BAFFR antibody or functional fragment thereof for use according to any one of embodiments C1-07, wherein the antibody or functional fragment thereof is to be administered to said subject every four (4) weeks (q4w).

C9. The anti-BAFFR antibody or functional fragment thereof for use according to any one of embodiments C1-07, wherein the antibody or functional fragment thereof is to be administered to said subject every two (2) weeks (q2w).

C10. The anti-BAFFR antibody or functional fragment thereof for use according to any one of embodiments C1-07, wherein the anti-BAFFR antibody or functional fragment thereof is to be administered on a monthly dosing regimen.

C11. The anti-BAFFR antibody or functional fragment thereof for use according to any one of embodiments C1-010, wherein the antibody or functional fragment thereof is to be administered subcutaneously to said subject.

C12. The anti-BAFFR antibody or functional fragment thereof for use according to any one of embodiments C1-011, wherein the anti-BAFFR antibody or functional fragment thereof is to be administered at a dose which comprises two unit doses of 150 mg the anti-BAFFR antibody or functional fragment thereof, and which is administered s.c. every four (4) weeks (q4w).

C13. The anti-BAFFR antibody or functional fragment thereof for use according to any one of embodiments C1-012, wherein the anti-BAFFR antibody or functional fragment thereof is to be administered in combination with an additional therapeutic agent.

C14. The anti-BAFFR antibody or functional fragment thereof for use according to embodiment C13, wherein the additional therapeutic agent is a steroid, e.g. a corticosteroid.

C15. The anti-BAFFR antibody or functional fragment thereof for use according to embodiment C14, wherein the additional therapeutic agent is prednisone.

C16. The anti-BAFFR antibody or functional fragment thereof for use according to any one of embodiments C14-C15, wherein prednisone is to be administered orally, at an amount of about 50 mg.

C17. The anti-BAFFR antibody or functional fragment thereof for use according to any one of embodiments C14-C16, wherein the additional therapeutic agent is to be administered prior to the administration of the first dose of the anti-BAFFR antibody or functional fragment.

C18. The anti-BAFFR antibody or functional fragment thereof for use according to any one of embodiments C14 to C17, wherein the additional therapeutic agent is to be administered prior to the administration of the first dose of the anti-BAFFR antibody or functional fragment and is not be administered for in combination with subsequent doses of the anti-BAFFR antibody or functional fragment.

C19. The anti-BAFFR antibody or functional fragment thereof for use according to any one of embodiments C1-018, wherein said subject achieves a sustained response as measured by ESSPRI or ESSDAI after the treatment with the anti-BAFFR antibody or functional fragment thereof.

C20. The method according to any one of preceding embodiments C1-019, wherein said antibody is an antibody which has demonstrated to be biosimilar to, or interchangeable to ianalumab.

Further Enumerated Embodiments

D1. A medicament for treating or preventing Sjögren's syndrome (SjS) in a subject in need of such treatment, said medicament comprising an anti-BAFFR antibody, wherein the dose of the anti-BAFFR antibody is from about 100 mg to about 300 mg.

D2. The medicament according to embodiment D1, wherein the dose of the anti-BAFFR antibody is from about 150 mg to about 300 mg.

D3. The medicament according to embodiment D1 or D2, wherein the said anti-BAFFR antibody includes heavy chain CDR1, CDR2 and CDR3 of SEQ ID Nos: 3, 4 and 5 respectively, and light chain CDR1, CDR2 and CDR3 of SEQ ID NOs: 6, 7 and 8, and wherein said antibody is to be administered to a subject in need thereof, as a dose of from about 50 mg to about 300 mg active ingredient.

D4. The medicament according to embodiment D1 or D2, wherein the said anti-BAFFR antibody includes a heavy chain sequence of SEQ ID NO: 9 and a light chain sequence of SEQ ID NO: 10.

D5. The medicament according to embodiment D1 or D2, wherein the dose of said antibody is of from about 150 mg to about 300 mg.

D6. The medicament according to any one of above embodiments, wherein the dose of said antibody is of about 150 mg active ingredient.

D7. The medicament according to any one of above embodiments, wherein the dose of said antibody is of about 300 mg active ingredient.

D8. The medicament according to any one of embodiments D1 to D7, wherein the antibody is to be administered to a subject in need thereof every four (4) weeks (q4w).

D9. The medicament according to any one of embodiments D1 to D7, wherein the antibody is to be administered to a subject in need thereof every two (2) weeks (q2w).

D10. The medicament according to any one of above embodiments, wherein the antibody is to be administered subcutaneously to a subject in need thereof.

D11. A use of an anti-BAFFR antibody for the treatment or prevention of Sjögren's syndrome in a subject in need thereof, wherein said anti-BAFFR antibody includes a heavy chain sequence of SEQ ID NO: 9 and a light chain sequence of SEQ ID NO: 10.

D12. A use of an anti-BAFFR antibody for the treatment or prevention of Sjögren's syndrome, wherein said anti-BAFFR antibody is ianalumab.

D13. A use of a liquid pharmaceutical composition comprising an anti-BAFFR antibody, a buffer, a stabilizer and a solubilizer, and means for subcutaneously administering the anti-BAFFR antibody to a subject having Sjögren's syndrome, for the manufacture of a medicament for the treatment of Sjögren's syndrome, wherein the said anti-BAFFR antibody includes heavy chain CDR1, CDR2 and CDR3 of SEQ ID NOs 3, 4 and 5 respectively, and light chain CDR1, CDR2 and CDR3 of SEQ ID NOs: 6, 7.

D14. A use of a liquid pharmaceutical composition comprising an anti-BAFFR antibody, a buffer, a stabilizer and a solubilizer, and means for subcutaneously administering the anti-BAFFR antibody to a subject having Sjögren's syndrome, for the manufacture of a medicament for the treatment of Sjögren's syndrome, wherein the said anti-BAFFR antibody includes a heavy chain sequence of SEQ ID NO: 9 and a light chain sequence of SEQ ID NO: 10.

D15. A use of a liquid pharmaceutical composition comprising an anti-BAFFR antibody, a buffer, a stabilizer and a solubilizer, and means for subcutaneously administering the anti-BAFFR antibody to a subject having Sjögren's syndrome, for the manufacture of a medicament for the treatment of primary Sjögren's syndrome, wherein the said anti-BAFFR antibody is ianalumab.

D16. The use according to any one of embodiments D11 to D15, wherein said antibody is at a dose of from about 50 mg to about 300 mg.

D17. The use according to embodiment D16, wherein said antibody is at a dose of about 300 mg.

D18. The medicament according to any one of above embodiments, or the use according to any one of above embodiments, wherein said antibody is an antibody which has demonstrated to be biosimilar to, or interchangeable to ianalumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

Abbreviations

AE adverse event

bid twice a day (for Latin: “bis in die”)

BMI Body Mass Index

CBC complete blood count

cm centimeter

CL/F the apparent systemic (or total body) clearance from plasma (or serum or blood) following administration (mass/volume)

CNS central nervous system

CV coefficient of variation

DMARDs disease-modifying antirheumatic drugs

ECG Electrocardiogram

eGFR estimated glomerular filtration rate

ELISA Enzyme-linked immunosorbent assay

EMG electromyography

EQ-5D EuroQual 5 dimensions (Standard instrument to measure the health-related quality of life)

ESSDAI EULAR Sjögren's Syndrome Disease Activity Index

ESSPRI EULAR Sjögren's Syndrome Patient Reported Index

EULAR European League against Rheumatism

FACIT-F Functional Assessment of Chronic Illness Therapy-Fatigue

FIH First in Human

h hour

HRCT high resolution computer tomography

i.v. intravenous

IA Interim analysis

INR International Normalized Ratio

kg kilogram

LC-MS/MS liquid chromatography/mass spectrometry-mass spectrometry

mAb monoclonal antibody

MCP-Mod Multiple Comparison Procedure—Modelling

MMRM Mixed effect Model Repeat Measurement

MRT mean residence time

NOAC Novel Oral Anti-Coagulant

NSAID Nonsteroidal Anti-Inflammatory Drug

PD Pharmacodynamic(s)

PhGA Physician global assessment scale

PK Pharmacokinetic(s)

PNS peripheral nervous system

PT prothrombin time

PTT partial thromboplastin time

qd once a day (for Latin “quaque die”)

QTcF QT interval corrected by Fridericia's formula

Racc Ratio of accumulation of drug

SAE serious adverse event

SjS Sjögren's Syndrome

SOM Site Operations Manual

SPT skin prick test

SS Safety Set

TEC tyrosine-protein kinase

Vz/F the apparent volume of distribution during the terminal elimination phase following administration (volume)

### EXAMPLE 1

### Preparing Anti-BAFFR Antibodies

To enable a person skilled in the art to practice the invention, the amino acid and nucleotide sequences of ianalumab are provided below.

Antibody ianalumab (MOR6654, or VAY736) binds specifically to BAFFR and is also described in international application published as W02010/007082. It is a human IgG1 kappa antibody obtained via phage display. Its heavy and light chains consist of SEQ ID NOs: 9 and 10. The Tables 1 and 2 below summarize the sequence characteristics of ianalumab.

TABLE 1
Brief description of the sequences listed
in the sequence listing of Table 2

| SEQ ID | |
|---|---|
| NO: | Description of the sequence |
| 1 | Amino acid sequence of the variable region (V_{H}) |
| | of the heavy chain of VAY736 |
| 2 | Amino acid sequence of the variable region (V_{L}) |
| | of the light chain of VAY736 |
| 3 | Amino acid sequence of HCDR1 of VAY736 |
| 4 | Amino acid sequence of HCDR2 of VAY736 |
| 5 | Amino acid sequence of HCDR3 of VAY736 |
| 6 | Amino acid sequence of LCDR1 of VAY736 |
| 7 | Amino acid sequence of LCDR2 of VAY736 |
| 8 | Amino acid sequence of LCDR3 of VAY736 |
| 9 | Amino acid sequence of the full length heavy chain of VAY736 |
| 10 | Amino acid sequence of the full length light chain of VAY736 |
| 11 | Nucleotide sequence encoding SEQ ID NO: 1 |
| 12 | Nucleotide sequence encoding SEQ ID NO: 2 |
| 13 | Human BAFFR amino acid sequence |
| 14 | Full length nucleotide sequence (including leader sequence and |
| | constant part) of MOR6654 heavy chain; nt 1-57 = leader; |
| | nt 58-429 = VH; nt 430-1419 = constant region (hIgG1) |
| 15 | Full length nucleotide sequence (including leader sequence and |
| | constant part) of MOR6654 light chain; nt 1-60 = leader; |
| | nt 61-384 = VL; nt 385-705 = constant region (hkappa) |

TABLE 2
Sequence listing

| SEQ | |
|---|---|
| ID | |
| NO: | Amino acid or Nucleotide Sequence |
| 1 | QVQLQQSGPGLVKPSQTLSLTCAISGDSVSSNSAAWGWIRQSPGRGLEWLG |
| | RIYYRSKWYNSYAVSVKSRITINPDTSKNQFSLQLNSVTPEDTAVYYCARYD |
| | WVPKIGVFDSWGQGTLVTVSS |
| 2 | DIVLTQSPATLSLSPGERATLSCRASQFISSSYLSWYQQKPGQAPRLLIYGS |
| | SSRATGVPARFSGSGSGTDFTLTISSLEPEDFAVYYCQQLYSSPMTFGQGTK |
| | VEIK |
| 3 | GDSVSSNSAAWG |
| 4 | RIYYRSKWYNSYAVSVKS |
| 5 | YDWWPKIGVFDS |
| 6 | RASQFISSSYLS |
| 7 | GSSSRAT |
| 8 | QQLYSSPMT |
| 9 | QVQLQQSGPGLVKPSQTLSLTCAISGDSVSSNSAAWGWIRQSPGRGLEWLGR |
| | IYYRSKWYNSYAVSVKSRITINPDTSKNQFSLQLNSVTPEDTAVYYCARYDW |
| | VPKIGVFDSWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 10 | DIVLTQSPATLSLSPGERATLSCRASQFISSSYLSWYQQKPGQAPRLLIYGS |
| | SSRATGVPARFSGSGSGTDFTLTISSLEPEDFAVYYCQQLYSSPMTFGQGTK |
| | VEIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVTK |
| | SFNRGEC |
| 11 | CAGGTGCAGCTGCAGCAGAGCGGCCCAGGCCTGGTCAAGCCCTCTCAGA |
| | CCCTGTCACTGACCTGCGCCATTTCAGGCGACAGCGTGAGCAGCAACAG |
| | CGCCGCCTGGGGCTGGATCAGGCAGAGCCCCGGTAGGGGCCTGGAATGG |
| | CTGGGCAGGATCTACTACAGGTCCAAGTGGTACAACAGCTACGCCGTGA |
| | GCGTGAAGAGCAGGATCACCATCAACCCTGACACCAGCAAGAACCAGTT |
| | CTCACTGCAGCTCAACAGCGTGACCCCCGAGGACACCGCCGTGTACTAC |
| | TGCGCCAGATACGACTGGGTGCCCAAGATCGGCGTGTTCGACAGCTGGG |
| | GCCAGGGCACCCTGGTGACCGTGTCAAGC |
| 12 | GATATCGTGCTGACACAGAGCCCCGCCACCCTGAGCCTGAGCCCAGGCG |
| | AGAGGGCCACCCTGTCCTGCAGGGCCAGCCAGTTTATCAGCAGCAGCTA |
| | CCTGTCCTGGTATCAGCAGAAGCCCGGCCAGGCCCCTAGACTGCTGATC |
| | TACGGCAGCTCCTCTCGGGCCACCGGCGTGCCCGCCAGGTTCAGCGGCA |
| | GCGGCTCCGGCACCGACTTCACCCTGACAATCAGCAGCCTGGAGCCCGA |
| | GGACTTCGCCGTGTACTACTGCCAGCAGCTGTACAGCTCACCCATGACC |
| | TTCGGCCAGGGCACCAAGGTGGAGATCAAG |
| 13 | MRRGPRSLRGRDAPAPTPCVPAECFDLLVRHCVACGLLRTPRPKPAGAS |
| | SPAPRTALQPQESVGAGAGEAALPLPGLLFGAPALLGLALVLALVLVGL |
| | VSWRRRQRRLRGASSAEAPDGDKDAPEPLDKVIILSPGISDATAPAWPP |
| | PGEDPGTTPPGHSVPVPATELGSTELVTTKTAGPEQQ |
| 14 | ATGGCCTGGGTGTGGACCCTGCCCTTCCTGATGGCCGCTGCCCAGTCAG |
| | TGCAGGCCCAGGTGCAGCTGCAGCAGAGCGGCCCAGGCCTGGTCAAGCC |
| | CTCTCAGACCCTGTCACTGACCTGCGCCATTTCAGGCGACAGCGTGAG |
| | CAGCAACAGCGCCGCCTGGGGCTGGATCAGGCAGAGCCCCGGTAGGGGC |
| | CTGGAATGGCTGGGCAGGATCTACTACAGGTCCAAGTGGTACAACAGCT |
| | ACGCCGTGAGCGTGAAGAGCAGGATCACCATCAACCCTGACACCAGCAA |
| | GAACCAGTTCTCACTGCAGCTCAACAGCGTGACCCCCGAGGACACCGCC |
| | GTGTACTACTGCGCCAGATACGACTGGGTGCCCAAGATCGGCGTGTTCG |
| | ACAGCTGGGGCCAGGGCACCCTGGTGACCGTGTCAAGCGCCAGCACCAA |
| | GGGCCCCAGCGTGTTCCCCCTGGCCCCCAGCAGCAAGAGCACCAGCGG |
| | CGGCACAGCCGCCCTGGGCTGCCTGGTGAAGGACTACTTCCCCGAGCCC |
| | GTGACCGTGTCCTGGAACAGCGGAGCCCTGACCTCCGGCGTGCACACCT |
| | TCCCCGCCGTGCTGCAGAGCAGCGGCCTGTACAGCCTGTCCAGCGTGGT |
| | GACAGTGCCCAGCAGCAGCCTGGGCACCCAGACCTACATCTGCAACGTG |
| | AACCACAAGCCCAGCAACACCAAGGTGGACAAGAGAGTGGAGCCCAAGA |
| | GCTGCGACAAGACCCACACCTGCCCCCCCTGCCCAGCCCCAGAGCTGCT |
| | GGGCGGACCCTCCGTGTTCCTGTTCCCCCCCAAGCCCAAGGACACCCTG |
| | ATGATCAGCAGGACCCCCGAGGTGACCTGCGTGGTGGTGGACGTGAGCC |
| | ACGAGGACCCAGAGGTGAAGTTCAACTGGTACGTGGACGGCGTGGAGGT |
| | GCACAACGCCAAGACCAAGCCCAGAGAGGAGCAGTACAACAGCACCTAC |
| | AGGGTGGTGTCCGTGCTGACCGTGCTGCACCAGGACTGGCTGAACGGCA |
| | AGGAATACAAGTGCAAGGTCTCCAACAAGGCCCTGCCAGCCCCCATCGA |
| | AAAGACCATCAGCAAGGCCAAGGGCCAGCCACGGGAGCCCCAGGTGTAC |
| | ACCCTGCCCCCCTCCCGGGAGGAGATGACCAAGAACCAGGTGTCCCTGA |
| | CCTGTCTGGTGAAGGGCTTCTACCCCAGCGACATCGCCGTGGAGTGGGA |
| | GAGCAACGGCCAGCCCGAGAACAACTACAAGACCACCCCCCCAGTGCTG |
| | GACAGCGACGGCAGCTTCTTCCTGTACAGCAAGCTGACCGTGGACAAGT |
| | CCAGGTGGCAGCAGGGCAACGTGTTCAGCTGCAGCGTGATGCACGAGGC |
| | CCTGCACAACCACTACACCCAGAAGAGCCTGAGCCTGTCCCCCGGCAAG |
| 15 | ATGAGCGTGCTGACCCAGGTGCTGGCTCTGCTGCTGCTGTGGCTGACCG |
| | GCACCAGATGCGATATCGTGCTGACACAGAGCCCCGCCACCCTGAGCCT |
| | GAGCCCAGGCGAGAGGGCCACCCTGTCCTGCAGGGCCAGCCAGTTTATC |
| | AGCAGCAGCTACCTGTCCTGGTATCAGCAGAAGCCCGGCCAGGCCCCTA |
| | GACTGCTGATCTACGGCAGCTCCTCTCGGGCCACCGGCGTGCCCGCCAG |
| | GTTCAGCGGCAGCGGCTCCGGCACCGACTTCACCCTGACAATCAGCAGC |
| | CTGGAGCCCGAGGACTTCGCCGTGTACTACTGCCAGCAGCTGTACAGCT |
| | CACCCATGACCTTCGGCCAGGGCACCAAGGTGGAGATCAAGCGTACGGT |
| | GGCCGCTCCCAGCGTGTTCATCTTCCCCCCCAGCGACGAGCAGCTGAAG |
| | AGCGGCACCGCCAGCGTGGTGTGCCTGCTGAACAACTTCTACCCCCGGG |
| | AGGCCAAGGTGCAGTGGAAGGTGGACAACGCCCTGCAGAGCGGCAACA |
| | GCCAGGAGAGCGTCACCGAGCAGGACAGCAAGGACTCCACCTACAGCCT |
| | GAGCAGCACCCTGACCCTGAGCAAGGCCGACTACGAGAAGCATAAGGTG |
| | TACGCCTGCGAGGTGACCCACCAGGGCCTGTCCAGCCCCGTGACCAAGA |
| | GCTTCAACAGGGGCGAGTGC |

### EXAMPLE 2

### Single Dose Trial in Primary Sjögren's Syndrome Patients

CVAY736X2201 is a randomized, placebo-controlled, single center, double-blind clinical trial. Twenty-seven (27) Sjögren's patients with active moderate-to-severe disease were enrolled in this study. At baseline, six patients received 3 mg/kg of VAY736 as single i.v. doses, 12 received 10 mg/kg of VAY736 as single i.v. doses, and 9 received a single dose of a placebo infusion. The primary endpoint of ESSDAI was reduced within 12 weeks, but improvements did not reach clinical or statistical significance.

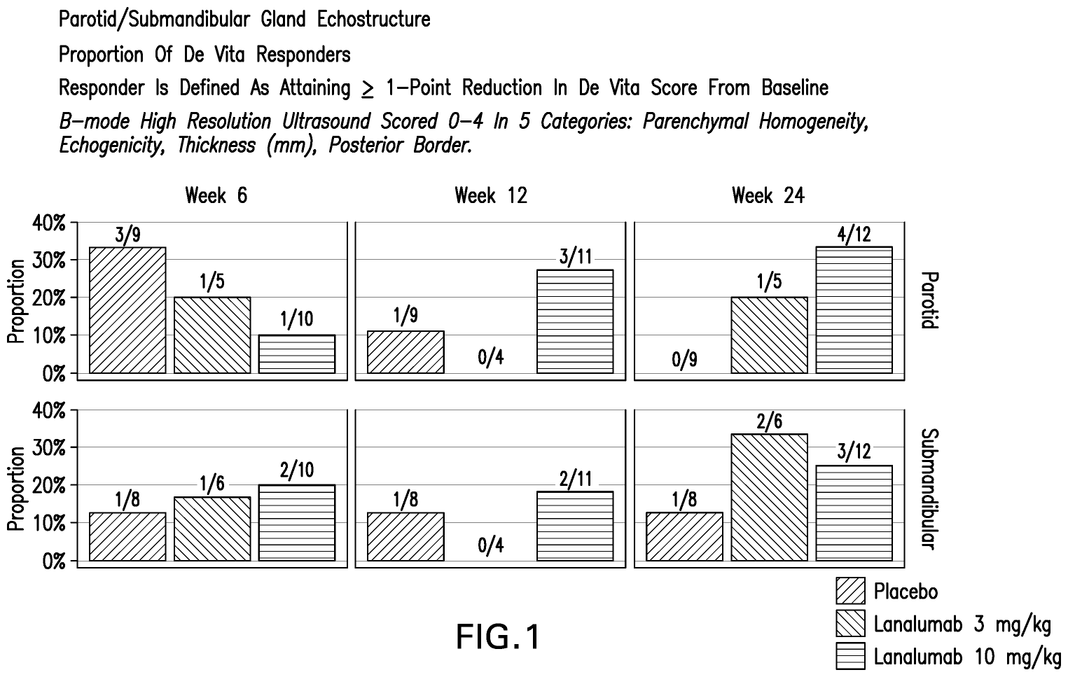

In pSS patients, early signs of salivary gland improvement in response to an effective intervention are detectable using a non-invasive, comprehensive, ultrasound-based approach over multiple time points. Salivary gland ultrasound (SGUS) multi-modal findings in this single dose ianalumab study showed evidence suggesting improved echo-structure (see FIG. 1: Parotid/submandibular gland echostructure), decreased inflammation and swelling.

There was variability between the two ianalumab dose groups in the clinical outcomes of ESSDAI, ESSPRI, MFI and patient and physician global assessments. In some outcomes, the effect of 3 mg/kg ianalumab appeared transient, with early signs of improvement at week 6 returning back towards baseline by week 12 or 24. In contrast, patients receiving 10 mg/kg ianalumab showed sustained effects up to week 24. These observations were in accordance with the observed ianalumab exposure, that is, ianalumab quantifiable levels detected approximately up to 8-12 weeks and to 12-16 weeks for the 3 mg/kg and 10 mg/kg dose groups, respectively.

### EXAMPLE 3

### PK PD Data

Level of receptor occupancy (RO) data have been evaluated. PK and PD data from RA and Sjögren's patients were used to establish the dose-exposure-response relationship for circulating B cells in blood. A two-compartmental population PK model with linear clearance was fitted to i.v. and s.c. data. Then, a PK/PD (B cells) model was fitted on the pooled RA/Sjögren's dataset using a sequential approach (i.e., by fixing PK parameters from the population PK model). Furthermore, a hypothesis-driven tissue RO model was developed. The model considers the competitive binding between VAY736 and soluble BAFF (sBAFF) on BAFF-R. Based on the above, it has been proposed that 300 mg s.c. to fully occupy BAFF-R in tissues (i.e. ≥90%) over the monthly dosing interval, 50 mg and 150 mg s.c. q4w to achieve tissue RO of about 80% in at least 50% of the patients. Doses 50 mg, 150 mg and 300 mg of ianalumab, s.c., administered q4w, provide evidence of increased efficacy due to targeting the BAFF-R pathway, in addition to the expected clinical benefit due to complete depletion of circulating B cells.

### EXAMPLE 3

A randomized, double-blind, placebo-controlled multicenter phase 2 dose-ranging study to assess the safety and efficacy of multiple ianalumab doses administered subcutaneously in patients with moderate to severe primary Sjögren's Syndrome

Methods: 190 patients with pSS were randomized 1:1:1:1 to monthly s.c. administrations of either placebo or one of three ianalumab doses, 5 mg, 50 mg and 300 mg. First-dose premedication was with 250 mg i.v. methylprednisolone. To be eligible, patients had to fulfill American European Consensus Group (AECG) criteria for pSS, be anti-Ro/SSA positive, have an ESSDAI ≥6 (on 7 of 12 domains: glandular, articular, lymphadenopathy, constitutional, cutaneous, hematologic and biologic), and European Sjögren's Syndrome Patient Reported Index (ESSPRI) ≥5. Statistical methods included MCP-Mod to assess the dose-response on change of ESSDAI (12 domains) from baseline and responder analysis to calculate the proportion of patients with points improvement on ESSDAI as secondary analysis.

Secondary endpoints included ESSPRI, Functional Assessment of Chronic Illness Therapy—Fatigue (FACIT-F), Physician's (PhGA) and Patient's Global Assessments (PaGA), SF-36, stimulated salivary flow (sSF) and Schirmer's test.

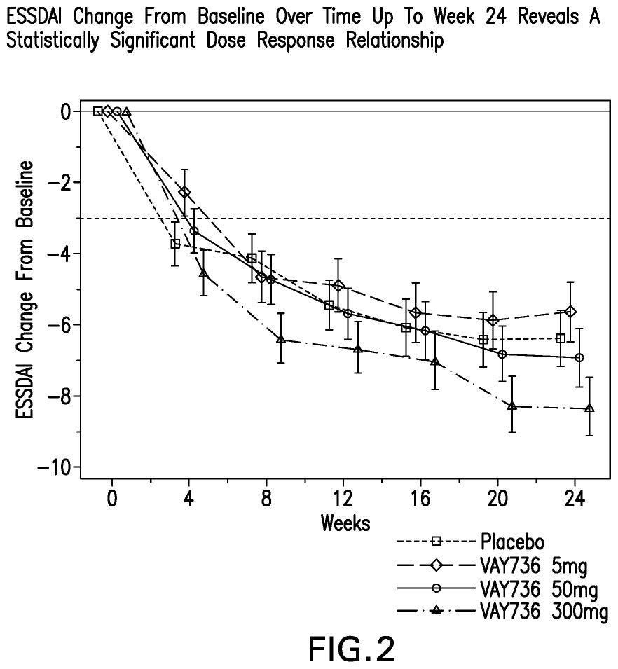

Results: The primary objective of the study was met. A statistically significant dose response was seen for ESSDAI as the primary endpoint (FIG. 2). The largest reduction in ESSDAI was 1.92 points over placebo for ianalumab 300 mg at Week 24. Secondary analysis on ESSDAI revealed for 300 mg versus placebo responder rates of 42/47 (89.4%) versus 30/49 (61.2%), a difference of 28.1% (p=0.0019), while no differences were seen for 5 mg and 50 mg versus placebo. Consistent with this result, change from baseline of PhGA was significantly different between ianalumab 300 mg and placebo (p=0.022). A numerical trend for improvement of sSF for ianalumab 300 mg compared to placebo was notable at Week 24 (p=0.092). However, the secondary efficacy endpoints ESSPRI and FACIT-F showed no benefits over placebo for improvements in burden of illness. Placebo responses were generally high. Incidence of treatment emergent adverse events were comparable across placebo and active groups, whereby local injection reactions were most frequent, mostly mild and showed a dose-response. The mean changes in ESSDAI over time showed decreases in all groups, including placebo, from Weeks 4 to 24. The mean changes with ianalumab 5 and 50 mg differed little from those of placebo, but with 300 mg showed a clear separation from Week 8 onwards, giving clear signs of efficacy. Stimulated salivary flow was significantly increased with 300 mg at Week 24, rising by 0.22 mL/min above placebo (0.01, 0.38, 95% Cl, p=0.037), with changes visible at Week 12, but unstimulated flow was unchanged. Tear flow (right and left eyes) showed trends towards an increase.

Despite the many challenges in planning reliable clinical trials in Sjögren's syndrome (selecting appropriate patients and endpoints, reliance on reported outcomes, poor methods for assessing tear and saliva production, etc.), as indicated by a legacy of disappointing results with B cell depletion, the current clinical trial was successful and showed several dose-related responses. The study showed that 300 mg ianalumab is a safe and effective dose, and that depleting BAFF-positive B cells can lower disease activity and raise saliva flow.

The study shows how pharmacokinetic/pharmacodynamic modelling can help to identify efficacious exposures, regimens to achieve these exposures, dose ranges for testing.

What is claimed is:

1. A method of treating or preventing Sjögren's syndrome in a subject in need thereof, comprising administering to said subject a therapeutically effective amount of an anti-BAFFR antibody or a functional fragment thereof.

2. The method according to claim 1, wherein the anti-BAFFR antibody or functional fragment thereof is ianalumab.

3. The method according to any claim 1 or 2, wherein the anti-BAFFR antibody or functional fragment thereof is to be administered at a dose of from about 50 mg to about 300 mg.

4. The method according to any one of claims 1 to 3, wherein the anti-BAFFR antibody or functional fragment thereof is administered at a dose of from about 150 mg to about 300 mg.

5. The method according to claim 4, wherein the anti-BAFFR antibody or functional fragment thereof is administered at about 300 mg.

6. The method according to claim 4, wherein the anti-BAFFR antibody or functional fragment thereof is administered at about 200 mg.

7. The method according to claim 4, wherein the anti-BAFFR antibody or functional fragment thereof is administered at about 150 mg.

8. The method according to any one of claims 1 to 7, wherein the anti-BAFFR antibody or functional fragment thereof is to be administered subcutaneously.

9. The method according to any one of claims 1 to 8, wherein the anti-BAFFR antibody or functional fragment thereof is to be administered on a monthly dosing regimen.

10. The method according to any one of claims 2 to 9, wherein the anti-BAFFR antibody or functional fragment thereof is to be administered at a dose which comprises two unit doses of 150 mg ianalumab, and which is administered s.c. every four (4) weeks (q4w).

11. The method according to any one of claims 1 to 10, wherein the anti-BAFFR antibody or functional fragment thereof is to be administered in combination with an additional therapeutic agent.

12. The method according to claim 11, wherein the additional therapeutic agent is a steroid, e.g. a corticosteroid.

13. The method according to claim 12, wherein the additional therapeutic agent is prednisone.

14. The method according to any one of claims 1 to 13, wherein the anti-BAFFR antibody or functional fragment thereof is to be administered in combination with about 50 mg prednisone, e.g. wherein prednisone is to be administered orally.

15. The method according to any one of claims 1 to 14, wherein said subject achieves a sustained response as measured by ESSPRI or ESSDAI after the treatment with the anti-BAFFR antibody or functional fragment thereof.

16. The method according to any one of claims 1 to 15, wherein said antibody is an antibody which has demonstrated to be biosimilar to or interchangeable to ianalumab.
